(19) United States
(12) Patent Application Publication
(10) Pub. No.: US 2014/0363473 A1
(43) Pub. Date: Dec. 11, 2014

(54) METHOD FOR PREPARING SPHEROIDS OF HUMAN PRIMARY HEPATOCYTES

(71) Applicant: SWISSCI AG, Neuheim (CH)

(72) Inventors: Jörg-Matthias Pollok, Hamburg (DE); Jeanette Bierwolf, Hamburg (DE)

(21) Appl. No.: 14/365,371

(22) PCT Filed: Dec. 14, 2012
(86) PCT No.: PCT/EP2012/075548

§ 371 Date: Jun. 13, 2014

(30) Foreign Application Priority Data
Dec. 16, 2011 (DE) 10 2011 121 317.5

(51) Int. Cl. A61K 35/407; C12N 5/071

(52) U.S. Cl. CPC A61K 35/407; C12N 5/0671
USPC 424/400; 424/93.7; 435/370

(57) ABSTRACT

The present invention relates to a method for preparing spheroids of human primary hepatocytes. The method comprises culturing of isolated human primary hepatocytes on a polysaccharide scaffold under conditions that allow the formation of hepatocyte spheroids, and subsequently dissolving the polysaccharide scaffold to release the hepatocyte spheroids. The spheroids obtained by the method of the invention are particularly suitable for being transplanted into a subject afflicted with a liver disease.

The present invention relates to a method for preparing spheroids of human primary hepatocytes. The method comprises culturing of isolated human primary hepatocytes on a polysaccharide scaffold under conditions that allow the formation of hepatocyte spheroids, and subsequently dissolving the polysaccharide scaffold to release the hepatocyte spheroids. The spheroids obtained by the method of the invention are particularly suitable for being transplanted into a subject afflicted with a liver disease.

### BACKGROUND

Orthotopic liver transplantation is the most applied curative treatment for liver based metabolic disorders or hepatic failure. Due to organ shortage, there is a strong need to develop alternative treatments that support or restore normal liver function. Interestingly, only small amounts (5% to 10%) of transplanted liver tissue are capable of providing sufficient function to correct a metabolic liver defect, and even only 1% to 5% of liver mass is needed for regeneration in patients with liver failure (Puppi et al. (2009), Methods Mol Biol 481, 1; Pietrosi et al. (2009), World J Gastroenterol 15, 2074).

Single cell suspensions have been contemplated in the art for liver cell transplantation, and this approach has been used in more than 80 case reports from different centers (Fitzpatrick et al. (2009), J Intern Med 266, 339). Unfortunately, limited success was accomplished in most liver diseases due to low cell engraftment rates. Data from rat model demonstrate that in most transplantation approaches, only 0.5% of the transplanted hepatocytes finally engraft into the recipient liver and relative little is known about long term engraftment of transplanted hepatocytes in humans (Allen et al. (2001) J Lab Clin Med 138, 298). In a case of an infant with Refsum's disease, eight intraportal infusions with single hepatocytes resulted in no more than 0.25% engraftment (Sokal et al. (2003), Transplantation 76, 735). Problems related to low engraftment and repopulation rates in the recipient livers represent the major barrier to successful treatment of liver disease by hepatocyte transplantation in humans.

The transplantation of three-dimensional (3D) scaffolds on which hepatocytes have been cultured is another alternative that has been contemplated in the art to overcome the low cell engraftment rates observed in transplantation approaches with single cell suspensions. This approach is based on the assumption that due to improved stability and stress-resistance, three-dimensional hepatic microtissues are more likely to engraft into diseased liver tissue. For example, poly(L-lactic acid) (PLLA) polymers have been used as scaffolds for hepatic tissue engineering (Török et al. (2011) Liver Transpl. 17, 104-114.). These scaffolds are transplanted into the recipient and are slowly degraded within a certain time after transplantation. However, scaffold-based transplantation is often known to be associated with an insufficient blood supply of the transplanted hepatocytes and with an undesirable bile removal.

Thus, there is a need for new therapeutic approaches for the curative treatment of liver disorders or hepatic failures. The present invention provides a method for culturing isolated human hepatocytes, preferably human primary hepatocytes, to three-dimensional liver microtissues. These liver microtissues, which are commonly referred to as hepatocyte spheroids, are particularly suitable for engraftment into diseased and/or dysfunctional liver tissue of a recipient. The method of the invention provides isolated hepatocyte spheroids which are free from any artificial scaffold or carrier, such as PLLA polymers. According to the method of the invention, hepatocytes are cultured on a polysaccharide scaffold which is dissolved after the spheroid formation. After dissolving the polysaccharide scaffold, intact hepatocyte spheroids can be harvested for downstream in vivo or in vitro uses, e.g. for toxicity studies or transplantation into a recipient. In this way, the method of the invention circumvents the disadvantages that have been reported in connection with single cell infusion and matrix implantation. The isolated spheroids are associated with high cell engraftment rates and rapid integration into the recipient liver tissue, thereby providing substantial aid in correcting metabolic liver defects and fulminant liver failure.

### DISCLOSURE OF THE INVENTION

The present invention provides a method for preparing three-dimensional aggregates of human hepatocytes, preferably human primary hepatocytes, which are particularly suitable for being used in transplantation medicine.

Thus, in a first aspect the present invention provides a method for preparing a spheroid of cultured human primary hepatocytes, comprising the steps of:
(a) culturing human primary hepatocytes on a polysaccharide scaffold under conditions that allow the formation of a hepatocyte spheroid;
(b) dissolving the polysaccharide scaffold to release the hepatocyte spheroid;
(c) separating the hepatocyte spheroid from the culture medium.

The method of the invention is directed to the preparation of liver spheroids that comprise or consist of metabolically vital human hepatocytes and are particularly useful for long-term support of liver functions after transplantation. As used herein, “hepatocyte spheroids” refer to three-dimensional, spheroidal cell aggregates of human hepatocytes which are interconnected by intercellular junctions, such as tight junctions. The presence of intracellular junctions can be detected by numerous methods known in the art, e.g. by transmission electron microscopy. Alternatively, the occurrence of tight junctions can be verified by detection of the zonula occludens protein 1 (Zo-1). The aggregates obtained by the method of the invention have a size which renders them suitable for being used in transplantation via portal infusion. Preferably, the diameter of a spheroid is at least 50 μm, at least 100 μm, at least 150 μm, at least 200 μm, or more.

The spheroids are cultured on the surface and/or within the pores of polysaccharide scaffolds. The method for preparing the transplantable spheroids of the invention comprises as a first step the seeding of isolated human primary hepatocytes to a polysaccharide scaffold. As used herein, “primary” hepatocytes are hepatocytes that have been isolated from the liver of a human by methods known in the art. For example, the hepatocytes can be derived from an explanted healthy donor organ. Alternatively, primary hepatocytes may also be obtained from an explanted organ of a subject that suffers from a liver disease; in the latter case, however, it is preferred that the hepatocytes to be used in the method according to the invention are taken from functional liver tissue, i.e. from a part of the liver that is not affected by the disease.

In a still further embodiment, the human primary hepatocytes are obtained by liver biopsy. For example, liver biopsy may be performed during abdominal surgery by removing tissue samples from one or more sites of the liver. Alternatively, a biopsy can be carried out transvenously through the blood vessels, or percutaneously by use of a hollow needle which is passed through the skin into the liver. CT scan or ultrasound images can be used to guide the needle during biopsy. Alternatively, a laparoscopic liver biopsy may be performed to harvest the human primary hepatocytes which are required for the method of the invention. In a preferred embodiment of the invention, the human hepatocytes that are used for seeding the polysaccharides have been obtained from a living human donor by liver biopsy.

The tissue samples obtained from biopsy are normally perfused with a suitable buffer immediately after removal from the organ. Suitable buffers that can be used for tissue perfusion include Custodiol® HTK Solution, University of Wisconsin solution (UW solution) or other solutions described in the art in connection with organ perfusion. Subsequently, the tissue samples are usually processed to obtain a single liver cell suspension. This can be achieved, for example, by a collagenase digestion procedure, e.g. the two step procedure described in Dandri et al. (2001), Hepatology 33, 981. The hepatocytes which are released from the tissue samples can be filtered and washed with a suitable buffer, such as Hepatocyte Wash Medium (Invitrogen), and either immediately used for seeding the polysaccharide scaffold or flash frozen in liquid nitrogen and stored at −80° C. until further use.

A particular advantage of the method of the present invention resides in the possibility to obtain human hepatocytes directly from the subject that is in need of hepatocyte cell transplantation. In such an embodiment, the spheroids produced by the method of the invention are derived from primary hepatocytes which are autologous to the patient, which has the particular advantage that immune suppression after transfer of the hepatocyte aggregates into the recipient, which is regularly required after transplantation of allogeneic cells, can be avoided. The transplantation of autologous spheroids is particularly helpful in cases where a patient is afflicted with a liver disease that has lead to the destruction of certain areas of the liver while other regions of the organ are still populated by functional liver tissue. In these cases hepatocytes from functional regions of the organ can be obtained by liver biopsy, and these cells can be used as a starting material for the method described herein. Liver defects in which the liver still maintains functional and vital hepatic tissue include, for example, chronic non-infectious liver diseases, like liver cirrhosis and metabolic liver diseases.

Alternatively, the hepatocyte spheroids of the invention can also be obtained from a human donor other than the subject to be treated by transplantation of the hepatocyte spheroids. In such an approach, the primary hepatocytes which serve as a starting material for the method of the invention are derived from a donor subject that is not genetically identical to the recipient subject receiving the spheroids (allogeneic transplantation). Where the spheroids to be transplanted are allogenic to the recipient, chronic immunosuppression is normally required to avoid rejection of the transplanted cells in the recipient. Immunosuppressive agents that may be administered to the subject receiving an allogenic hepatocyte spheroid of the invention include, e.g., corticosteroids, antimetabolites such as methotrexate, azathioprine, leflunomide, cyclosporine, tacrolimus, sirolimus, everolimus, mycophenolate mofetil, and antibodies such as muromonab-CD3 and rituximab.

The primary hepatocytes obtained from the donor and processed as indicated above are then seeded to a polysaccharide scaffold and cultured to spheroids. This step is preferably performed ex vivo, i.e. outside the human body. As used herein, a scaffold or carrier is a three-dimensional polymer matrix which supports cell attachment and enables the formation of intercellular contacts between the cells that are seeded to said matrix. The terms “scaffold” and “carrier” will be used interchangeably herein. Preferably, the scaffold or carrier is a porous material that allows cells seeded thereto to grow on the surface and/or within the pores. The number of cells that are seeded to the polysaccharide scaffold will depend on different factors, such as the specific material and the size of the scaffold to which the hepatocytes are inoculated, the conditions used in the subsequent culturing of the hepatocytes, and the age of the cells used for seeding the scaffolds. Where freshly isolated primary hepatocytes are used for seeding, the number of cells used for seeding a single scaffold having a diameter of about 15-20 mm and a thickness of 1-2 mm will be in the range of about 1×10⁴, 5×10⁴, 1×10⁵, 5×10⁵, 1×10⁶, 5×10⁶, 1×10⁷, 5×10⁷, 1×10⁸, 5×10⁹, or more. Preferably, the number of cells used for seeding a scaffold of this size will be in the range of 1×10⁶ to 1×10⁸, more preferably 1×10⁶. Inoculation is achieved, e.g. by suspending a predetermined number of isolated hepatocytes in an appropriate volume of a buffer (e.g. 200-400 μl) and applying the solution to the scaffold.

The polysaccharide scaffold for use in the preparation of transplantable spheroids can be any polysaccharide material that has been described in the field of tissue engineering as a matrix to which cells can attach. Preferably, the polysaccharide scaffold is highly porous in nature, thereby offering a favourable microenvironment for the formation of the spheroids in the interstices of the pores. The scaffold to be used according to the invention is composed of a material that can be dissolved under mild conditions which do not adversely affect the viability and structural integrity of the spheroids that have formed on the surface and/or within the pores of the scaffolds. Suitable materials include common gelling agents, e.g., alginate, agar, carrageenan, processed euchema seaweed, locust bean gum, guar gum, tragacanth, acacia gum, xanthan gum, tara gum, gellan, pectin and celluloses (e.g. methylcellulose). Other polysaccharides that can be dissolved under mild conditions can also be used.

In a particularly preferred embodiment, the scaffold for culturing the hepatocyte spheroids of the invention comprises or consists of alginate. As used herein, alginate refers to a salt or ester of the heteropolysaccharide alginic acid, the latter of which consists of repeating units of β(1-4)D-mannuronic acid and α(1-4)L-guluronic acid. The scaffold can comprise or consist of, for example, a sodium, potassium, calcium, or ammonium salt of alginic acid. Alginate scaffolds for tissue engineering are commercially available, e.g., the AlgiMatrix™ 3D Culture System from Invitrogen (Carlsbad, USA).

The isolated hepatocytes that have been seeded on the scaffolds are in a subsequent cultured under conditions that allow the formation of the hepatocyte spheroids. Specifically, a culture medium suitable for growing human hepatocytes will be used. Cell culture media adapted to the requirements of human hepatocytes are known in the art and include, for example, Williams' Medium E (Invitrogen, Carlsbad, USA), Hepatocyte Culture Medium (BD Bioscience, Heidelberg, Germany), Basal HepaRG Medium (3H Biomedical AB, Uppsala, Sweden), HBM Basal Medium (Lonza Cologne GmbH, Cologne, Germany), or Hepatocyte Basal Medium (United States Biological, Swampscott, USA). The media can be supplemented with further compounds, such as growth factors, buffers, antibiotics and the like in order to optimize spheroid formation on the particular scaffolds.

For example, a culture medium that has been proven particularly useful is Williams' Medium E that has been supplemented with the following ingredients: 200 mM low endotoxin L-alanyl-L-glutamine (Biochrom, Berlin, Germany), 1M HEPES buffer (Biochrom), 100 mM sodium pyruvate (Invitrogen), 4 μg/mL of insulin (Invitrogen), 5 nM dexamethasone (Sigma-Aldrich, St. Louis, Mo.), 10 ng/mL epidermal growth factor (Invitrogen), 10 ng/mL recombinant human thrombopoietin (Cell Systems, St. Katharinen, Germany), 10 ng/mL recombinant human hepatocyte growth factor (Bachem, King of Prussia, USA), 10% heat-inactivated fetal bovine serum (Invitrogen), and 1% penicillin/streptomycin (Invitrogen). The culture medium that has been used in the below examples is described in Bierwolf et al. (2011), Biotechnol Bioeng.; 108(1):141-50.

Another culture medium that can be used for culturing the hepatocytes on the scaffolds is serum-free Williams' E medium (without l-glutamine) that is supplemented with 2 mM N-acetyl-l-alanyl-l-glutamine (Biochrom), a 20 mM 4-(2-hydroxyethyl)-1-piperazine ethanesulfonic acid buffer (Biochrom), 4 μg/mL insulin (Gibco BRL), 1 mM sodium pyruvate (Gibco BRL), 5 nM dexamethasone (Sigma, St. Louis, USA), 10 ng/mL epidermal growth factor (Gibco BRL), and 1% penicillin/streptomycin (Biochrom).

The scaffolds can be incubated under static conditions in a humidified atmosphere of about 5% CO₂ and 95% air in an incubator. The scaffolds can be cultured at a temperature between 30 and 40° C., preferably at a temperature between 33° C. and 38° C., and more preferably 37° C. The cell culture is maintained for a period of 1-21, preferably 3-10, and more preferably 5-7 days.

Alternatively, the seeded scaffolds may also be cultured in a recirculation bioreactor. To expose the hepatocytes to a recirculating medium flow, seeded scaffolds are fixed in a Cellmax Quad flow module perpendicularly to the flow vector (see Török et al. (2011), Liver Transplantation 17:104-114). For example, a pulsatile culture medium flow of 10-40 mL/minute, e.g. 24 mL/minute, can be used. Two-thirds of the volume of the re-circulated culture medium should be exchanged every other day during the entire culture period. The skilled person will readily be able to determine alternative culture conditions that can be used for growing the hepatocytes seeded on the scaffolds to three-dimensional hepatocyte spheroids.

After the hepatocyte spheroid on the scaffold has reached its pre-determined size and state of differentiation, the scaffold is dissolved in order to release the spheroids into the culture medium. The point in time at which the scaffold is to be dissolved will vary dependent on the specific culture conditions used and the desired size and differentiation state of the spheroid. The differentiation state of the spheroid can be monitored, e.g. by examining spheroids in regular intervals by electron microscopy. The specific procedure that is applied for dissolving the scaffold depends on the scaffold material that has been used for culturing the hepatocytes. In one embodiment of the invention, enzymes are added to the scaffolds which degrade the scaffold while leaving the spheroids unaffected. Numerous enzymes have been described that specifically degrade polysaccharides, such as xanthan (Hashimoto et al. (2003), J Biol Chem, 278(9):7663-73), agar (Leon et al. (1992), Appl and Environ Microbiology 58 (12):4060-4063), cellulose (Lynd et al. (2002), Microbiol Mol Biol Rev, 66(3):506-77), pectin (Blanco et al. (1999), FEMS Microbiol Lett, 175(1):1-9), amongst others.

Where the polysaccharide scaffold consists of alginate, it is preferred to dissolve it by the addition of a chelating agent, such as citrate or EDTA. Alginate requires divalent cations such as Ca²⁺ for maintaining its polymerized structure. Deprivation of the cations by chelating agents such as EDTA results in the degradation of the alginate. For example, if the AlgiMatrix™ 3D Culture System from Invitrogen is used, a sodium citrate-containing or a versene-containing dissolving buffer can be used according to the recommendation of the manufacturer. The scaffolds are incubated for 5-30 minutes in the presence of an appropriate amount of the citrate or versene-containing buffer until the scaffolds have completely dissolved and the spheroids are completely released in the surrounding medium. The spheroids obtained by the dissolution step are free of the alginate scaffold that used to surround them during the culturing step.

Generally, care should be taken during the dissolution not to damage the hepatocyte aggregates that have been grown on the surface of the polysaccharide scaffold. It should be ensured that the scaffold material is dissolved without affecting the integrity or metabolic function of the tissue-like hepatocyte spheroid. For example, where the scaffold material is dissolved by the addition of citrate to the culture medium, the concentration of the citrate must not be so high that the hepatocytes which are attached to the scaffold material are lysed. For the skilled person, several methods known in the art are available for assessing the viability of the hepatocytes. As described in the below Example 3, it is for instance possible to determine the release of lactate dehydrogenase (LDH) from damaged cells to determine cell viability and to screen for toxicological effects caused by the reagents used for dissolving the carriers scaffolds.

The spheroids which are suspended in the solution after the carriers have been dissolved are then harvested by separating them from the culture medium. The spheroids can be harvested, e.g. by centrifugation at 200×g for 3-5 minutes. Alternatively, the spheroids can also be harvested by filtration through a filter material having a pore size that retains cell aggregates with a diameter of at least 50, 100 or 150 μm. The harvested spheroids can optionally be washed one or more times to remove any undesired substances that could interfere with the desired downstream use, e.g. transplantation. For example, the spheroids may be washed with Hepatocyte Wash Medium (Invitrogen, Carlsbad, USA) to remove cellular debris or substances which have been used for dissolving the carrier, such as enzymes and the like. The harvested spheroids can directly be used for being transplanted into the recipient or for in vitro studies or they can be flash frozen in liquid nitrogen and stored at −80° C. until further use.

In a further aspect, the invention relates to an isolated spheroid of human primary hepatocytes obtainable by the above-described method. Preferably, the isolated spheroid of human primary hepatocytes is obtained by
(a) culturing human primary hepatocytes on an alginate scaffold under conditions that allow the formation of a hepatocyte spheroid;
(b) dissolving the alginate scaffold, preferably by the addition of a chelating agent, such as citrate or EDTA, to release the hepatocyte spheroid;
(c) separating the hepatocyte spheroid from the culture medium, preferably by filtration or centrifugation.

The spheroid obtained by the above method is preferably for use in a method of liver cell transplantation as described elsewhere herein.

In a still further aspect, the invention relates to an isolated hepatocyte spheroid of cultured human primary hepatocytes, wherein said spheroid is free of any artificial scaffold material. This means that the spheroid is not attached to or otherwise associated with an artificial scaffold material that is commonly used in the field of tissue engineering, such as alginate, PLLA, and the like. As used herein, artificial scaffold materials refer to synthetic matrices which do not occur in natural liver tissue. The term is not meant to include, e.g. the naturally occurring extracellular matrix.

The hepatocyte spheroids of the invention show a well-preserved liver cell-specific functionality and morphology. For example, the hepatocytes within the spheroids are in functional attachment to each other, as demonstrated, e.g. by detection of the zonula occludens protein 1 (Zo-1). Zo-1 is a marker for tight junctions indicating bile canaliculi formation between adjacent hepatocytes in the liver. In a preferred embodiment, bile canaliculi between adjacent hepatocytes are present in at least 50%, 60%, 70%, 80%, 90%, 95% or more of the hepatocytes within the spheroid provided by the present invention.

Unlike cell aggregates that are provided by culturing cells obtained from established cell lines, the spheroids of the invention comprise or consist of highly differentiated hepatocytes that show an excellent metabolic profile which indicates that the hepatocytes support the detoxifying function of natural liver tissue. For example, the spheroids have maintained the ability to produce human albumin and α1-antitrypsin. In one preferred embodiment of the invention, the production of human α1-antitrypsin in the hepatocytes of the spheroid is at least 50%, more preferably 60%, 70%, 80%, 90% or even up to 95% or more of the production determined in freshly isolated human primary hepatocytes when tested under the same conditions, e.g. in an ELISA assay as the one described in Example 5 below, or in a nucleic acid-based detection assay, e.g. quantitative RT-PCR. Similarly, the production of human albumin in the hepatocytes of the spheroid of the invention is at least 50%, more preferably at least 60%, 70%, 80%, 90% or even up to 95% or more of the production that is determined in freshly isolated human primary hepatocytes tested under the same conditions. A method for determining the production of human albumin in hepatocytes is described, for example, in the below Example 4. However, other test assays may also be used for measuring albumin expression, e.g. quantitative RT-PCR.

The isolated spheroids provided by the present invention are particularly suitable for being used in the field of medicine, preferably transplantation medicine. Hepatocyte implantation approaches described in the prior art are commonly associated with low cell engraftment rates and marginal effects achieved in most liver diseases. Transplantation of the spheroids provided by the present invention result in highly functional tissue which becomes rapidly integrated into the recipient's liver. Therefore, transplantation of cellular spheroids as an alternative for single cells improves engraftment and repopulation rates in recipient livers.

As shown in the below examples, hepatocytes from the livers of three donors suffering from metabolic liver diseases were used for three-dimensional cell culturing on alginate scaffolds. The formation of spheroids was observed after 3 days of culture and spheroids approached their maximum diameter of nearly 100 μm at day 7. Immunohistological studies were conducted to examine cell-to-cell interactions and reorganization or neo tissue regeneration within the pores of the scaffolds. These studies revealed that the hepatocyte spheroids cultured on alginate scaffolds established a fine-structure which is typical for liver tissue as demonstrated by CK18 immunofluorescent staining. Furthermore, detection of actin filaments labeled by phalloidin and positive staining of Zo-1 revealed re-formation of bile canaliculi and bipolar configuration (see Example 7). The preservation of a polarized cell and membrane architecture is essential for biliary excretion and xenobiotic elimination. In addition, positive nuclear staining for hepatocyte specific transcription factor HNF-4 demonstrates the presence of highly differentiated cells within the new liver tissue (see Example 7). Furthermore, realtime-PCR studies provided evidence that the spheroids cultured on alginate scaffolds are composed of highly differentiated hepatocytes, although a loss of expression was observed compared to the native tissue. Taken together, these results demonstrate that the spheroids obtained according to the present invention are highly suitable for replacing and/or supplementing diseased liver tissue.

The spheroids provided by the invention have a diameter of at least 50, 100 or 150 μm. The size of the spheroids can be adapted by altering the culturing time. Under the conditions used in the below examples, spheroids with a size of about 100-150 μm were obtained after a culturing time of 7 days. If specific transplantations require hepatocyte aggregates smaller than 100 μm, it is readily possible to shorten the culturing time, e.g. to 3-4 days. Preferably, the size of the spheroids transplanted into the recipient will not exceed 150 μm so as to minimize the risk of portal thrombosis and donor cell embolization into the lungs.

Due to their capability to integrate into liver tissue and to provide liver tissue functions, the isolated spheroids of the invention are particularly suited for use in a method of treating a liver disease in a patient. The term “liver disease” as used herein refers to a wide variety of conditions which are characterized by an impaired function of the liver. Liver diseases which can be treated by transplantation of the hepatocyte spheroids of the present invention include, but are not restricted to, hepatitis A, B and C, liver cirrhosis, α1-antitrypsin deficiency, Wilson Disease, hemochromatosis, bile duct obstruction, glycogen storage disease, Reye's syndrome in young children, hereditary tyrosinemia type I, parasitic infections, primary sclerosing cholangitis, secondary sclerosing cholangitis, chronic Budd Chiari syndrome, polycystic liver disease, oxalosis, urea cycle defects, mitochondrial depletion syndrome, Alagille syndrome, Crigler-Najjar syndrome, primary familiar intrahepatic cholestasis, neonatal hepatitis, biliary atresia, fulminate hepatic failure, alcoholic cirrhosis, autoimmune hepatitis, overlap syndrome, liver intoxication due to paracetamol, phalloidine or other agents.

Transplantation of the hepatocyte spheroids of the invention can be achieved in different ways, for example, by transplantation into the spleen or pancreas, or by introduction directly into the liver, e.g., via portal infusion.

As outlined elsewhere herein, it is particularly preferred that the hepatocyte spheroids are derived from primary hepatocytes which are autologous to the patient. This means that primary hepatocytes are taken from the liver of a subject in order to prepare three-dimensional hepatocyte spheroids, and the ex vivo cultured spheroids are then transplanted back into the same patient. In this manner, rejection of the transplanted hepatocyte spheroids by the immune system of the recipient can be avoided.

Another particular advantage of the invention is the possibility to extend the availability of high quality hepatocyte aggregates for several days. In common cell transplantation approaches, there might not be an appropriate patient to receive the cells immediately upon removal of the primary hepatocytes from the donor organs. It has been shown in the art that cryopreservation of human hepatocytes impairs cell quality and successful engraftment of the cells into the liver tissue. According to the method of the present invention, the isolated primary hepatocytes are cultured for several days without any significant loss of their metabolic function, thereby extending the availability of highly functional cells for up to 7 days, up to 10 days or even more, after removal of the primary hepatocytes. Alternatively, the spheroids of the invention may also be frozen in liquid nitrogen and stored at temperatures between −20° C. and −80° C. until being used in transplantation.

The invention thus also relates to a method of transplanting a hepatocyte spheroid of cultured human hepatocytes into a recipient in need thereof, said method comprising
(a) culturing isolated human primary hepatocytes on a polysaccharide scaffold under conditions that allow the formation of a hepatocyte spheroid;
(b) dissolving the polysaccharide scaffold to release the hepatocyte spheroid;
(c) separating the hepatocyte spheroid from the culture medium; and
(d) transplanting the hepatocyte spheroid obtained from the above step (c) into the recipient.

The recipient being in need of such transplantation preferably is a subject which suffers from one of the liver disease mentioned above.

Another advantage that is associated with the hepatocyte spheroids of the invention is the possibility to obtain primary hepatocytes from a patient who suffers from a liver disease that is caused by a single gene defect. For example, metabolic liver diseases may be based on a single gene deficiency, while the liver otherwise functions normally. In these cases, the method of the invention can be applied in within a gene therapy approach. Specifically, a functional version of the abnormally mutated gene will be inserted into an unspecific or specific location of the genome of primary hepatocytes obtained from a patient. The hepatocytes modified in this manner are then used to prepare spheroids according to the above-described method of the invention. In a final step, the spheroids are then implanted into the patient in need of treatment, which preferably is the patient from which the hepatocytes haven been obtained.

For replacing the dysfunctional gene which causes the liver disease, an intact copy of said gene is cloned into a viral or non-viral vector. The gene will be operably linked to a promoter element and, optionally, to an enhancer element to ensure its expression in the hepatocytes. The vector will typically be a viral vector. Suitable viral vectors for use in the present invention are recombinant DNA or RNA viruses, more preferably replication-deficient viruses, and include, for example, detoxified retrovirus, adenovirus, lentivirus, adeno-associated virus (AAV), herpes virus, poxvirus, vaccinia virus, poliovirus, Sindbis virus, polyomavirus, such as simian virus 40 (SV40), human immunodeficiency virus (HIV), and others.

Adenoviruses are particularly preferred, as the transduction efficiency is typically higher with adenoviruses compared to other viruses. The adenovirus may be a human adenovirus type 5 (hAd5) vector, an E1-deleted and/or an E3-deleted adenovirus. For example, an adenoviral vector can be constructed by the rescue recombination technique as described in McGrory, et al. (1988), Virology 163:614-617. Briefly, the transgene of interest is cloned into a shuttle vector that contains a promoter, a polylinker and flanking adenovirus sequences from which E1A/E1B genes have been deleted. Suitable shuttle vectors include, e.g., the plasmid “pAC1” (McGrory, et al. (1988), Virology 163:614-617) which encodes portions of the left end of the human adenovirus 5 genome but which lacks the early protein region comprising E1A and E1B sequences that are essential for viral replication. Another suitable shuttle plasmid is “ACCMVPLPA” (Gomez-Foix et al. (1992), J. Biol. Chem. 267: 25129-25134) which contains a polylinker, CMV promoter and SV40 polyadenylation signal flanked by partial adenovirus sequences from which the E1A/E1B genes have been deleted. The shuttle plasmid can be co-transfected, e.g., by lipofection or calcium-phosphate-transfection, along with a plasmid comprising the entire human adenovirus 5 genome with a length that is too large to be encapsidated into suitable host cells (e.g., human 293 cells). In a subset of cells “rescue recombination” between the shuttle vector and the helper plasmid will occur, creating a plasmid which contains the gene of interest in the place of the E1A/E1B genes and of the additional sequences which previously rendered the plasmid too large to be encapsidated. This can be monitored, e.g., with the beta-galactosidase/x-gal system which is well known in the art. The resulting plasmid of interest will be small enough to be encapsidated but replication deficient (see, e.g., Giordano et al. (1996), Nature Medicine 2: 534-539).

Recombinant viral vectors can be plaque-purified according to standard techniques. For example, recombinant adenoviral vectors can be propagated in human 293 cells (which provide E1A and E1B functions in trans) to titers in the range of 10⁷-10¹³ viral particles/mL. Prior to in vivo application viral vectors may be desalted by gel filtration methods, such as Sepharose columns, and purified by subsequent filtering. Purification reduces potential deleterious effects in the subject to which the vectors are administered. The administered virus is substantially free of wild-type and replication-competent virus. The purity of the virus can be proven by suitable methods, such as PCR amplification.

Non-viral expression vectors may also be used for introducing a functional AGAT gene into a human subject. Suitable expression vectors permit the in vivo expression of the AGAT gene in the target cell. Examples for non-viral expression vectors include vectors such as cages (Niwa et al. (1991), Gene, 108: 193-200), pBK-CMV, pcDNA3.1, pZeoSV (Invitrogen, Stratagene). These vectors may be administered, for example, by direct injection or non-invasive catheter or injector methods. Alternatively, target cells that have been removed from a subject, for example, by a biopsy procedure, may be transfected with the vector construct in an ex vivo procedure. The cells can then be implanted into or otherwise administered to a subject, preferably into the subject from whom they were obtained. Suitable methods for the transfer of non-viral vectors into target cells are for example the lipofection method, calcium-phosphate co-precipitation method, DEAE-dextran method and direct DNA introduction methods using micro-glass tubes and the like. Prior to the introduction of the vector, the hepatocytes may be treated with a permeabilization agent, such as phosphatidylcholine, streptolysins, sodium caprate, decanoylcarnitine, tartaric acid, lysolecithin, Triton X-100, and the like.

The successful treatment of liver disease by gene therapy has already been demonstrated in the prior art. For example, Grossmann et al. (1994), Nat Genet 6, 335, described an ex vivo approach to gene therapy for familial hypercholesterolaemia. Briefly, a patient suffering from this disease was transplanted with autologous hepatocytes that had been genetically corrected with recombinant retroviruses carrying the LDL receptor. The patient tolerated the procedure well, and analysis of the liver tissue four months after therapy revealed evidence for engraftment of transgene expressing cells. The patient's LDL/HDL ratio declined from 10-13 before gene therapy to 5-8 following gene therapy, and these improvements remained stable for more than 18 months. Another disease that could be cured by gene therapy is inborne ornithine transcarbamylase deficiency (OTCD), a rare urea cycle disorder that is caused by a number of different mutations in the gene encoding the ornithine transcarbamylase.

According to a further aspect, the hepatocyte spheroids of the invention may also find use in toxicity studies. For example, the spheroids will be helpful to assess hepatotoxicity of a compound, such as a candidate drug. For this purpose, a candidate drug can be contacted with the hepatocyte spheroids of the invention in a concentration and under conditions that correspond to the situation in vivo after administration of the candidate drug into the patient. In general, a candidate drug will be contacted and incubated with the three-dimensional spheroids for a pre-defined incubation time, e.g. about 1-24 hours. After contacting the spheroid with the candidate drug, one or more liver-specific factors or parameters are measured, such as the expression of the HNF-4 transcription factor, and/or the expression of α1-antitrypsin. The results obtained from the measurements are compared to those obtained from spheroid controls that have not been contacted with the candidate drug. Any decrease in the expression of liver-specific factors may indicate a possible hepatotoxicity of the candidate drug. As hepatotoxicity often results from interaction of two or more different drugs, the spheroids of the invention can furthermore be used to study combinations of a candidate drug with other known drugs.

On the other hand, spheroids prepared from diseased liver tissue can be conveniently used as a model system in drug screening assays to analyze the efficacy of a candidate drug in curing or ameliorating the symptoms of the respective disease.

### BRIEF DESCRIPTION OF DRAWINGS

FIG. 1 shows the loss of hepatocytes during 14 days of culturing as measured by DNA content (FIG. 1A) and the LDH release from damaged cells (FIG. 1B).

FIG. 2 shows the results from measuring different hepatocyte factors in supernatants from spheroids cultured on alginate scaffolds. FIG. 2A depicts the concentration of human albumin in absolute figures, while FIG. 2B shows the concentration of human albumin in relation to the DNA content per scaffold. FIG. 2C shows the concentration of α1-antitrypsin. FIG. 2D shows the concentration of α1-antitrypsin in relation to the DNA content per scaffold. FIG. 2E shows the production of urea. FIG. 2F shows the production of urea in relation to the DNA content per scaffold.

FIG. 3 shows the gene expression results as determined by real-time PCR of hepatocytes cultured on alginate scaffolds compared to native tissue and freshly isolated cells. 1=tissue before isolation; 2=cells after isolation; 3=after 1 day of culturing; 4=after 7 days of culturing; 5=after 14 days of culturing.

FIG. 4 shows the gene expression as determined by real-time PCR of hepatocyte spheroids of the invention after transplantation into murine liver. 1=native liver tissue; 2=primary hepatocytes after isolation; 3=after 1 day of culturing; 4=after 7 days of culturing; 5=8 weeks after transplantation into mice.

### EXAMPLES

The present study was approved by the committee of the Ärztekammer Hamburg in accordance with national guidelines and the 1975 Declaration of Helsinki. It was conducted after having received informed consent in writing from the parents of each patient.

### Example 1

### Isolation of Primary Hepatocytes

Primary human hepatocytes were isolated from the livers of 3 individuals suffering from metabolic diseases who underwent liver transplantation. Two patients were afflicted by urea cycle disorders, whereas one patient suffered from primary oxalosis. Table 1 displays more details about the donor data.

TABLE 1
Details on patients from which primary human hepatocytes
were derived for in vitro testing

| | | | Time of | Cell |
|---|---|---|---|---|
| | | | cold ischemia | Viability |
| Patient | Donor age | Donor diagnosis | (hours) | (%) |
| A | 15 months | Ornithine | 30 | 99 |
| | | Transcarbamylase | | |
| | | Deficiency | | |
| B | 32 months | Carbamoyl Phosphate | 24 | 80 |
| | | Synthetase Deficiency | | |
| C | 18 years | Primary Oxalosis | 48 | 95 |

| | |
|---|---|
| Average viability ± Standard deviation (%) | 91.3 ± 8.2 |

Immediately after explanation, the liver tissue was perfused with cold (4° C.) Custodiol® HTK Solution (Dr. F. Köhler Chemie, Bensheim, Germany) and stored at 4° C. until the time of hepatocyte isolation. Samples of native tissues were flash frozen and stored at −80° C. before starting cell isolation procedure. A two step collagenase digestion was used as described before in Dandri et al. (2001), Hepatology 33, 981, to obtain a single liver cell suspension. The liver tissue was placed in a sterile glass bowl located in a waterbath at 37° C. A branch of the vena portae was cannulated and the liver tissue was perfused at 37° C. with a flow speed of 40 to 100 ml/min according to the size of the liver specimen. The first perfusion solution was a calcium free buffer with duration of 8 to 10 minutes. Thereafter perfusion was continued with a 0.05% collagenase solution (Worthington Collagenase Type II, Worthington) for 10 to 30 minutes. Finally, the digested liver tissue was placed in cold Hepatocyte Wash Medium (Invitrogen) and the liver capsule was incised. The hepatocytes were mobilized with gentle shaking of the tissue and the suspension was filtrated through a nylon mash with a pore size of 100 μm. After filtering hepatocytes were separated by centrifugation at 50×g for 5 minutes and washed 3 times with Hepatocyte Wash Medium. Cell number and viability were determined by Trypan blue test. Samples of cells were flash frozen in liquid nitrogen immediately after isolation procedure and stored at −80° C. for further analysis.

Results:

Human hepatocytes could be successfully isolated from metabolic diseased livers by the above procedure. A mean cell viability of 91.3±8.2% (n=3 isolations) was determined by Trypan blue test immediately after hepatocyte isolation from the livers. Especially isolations from the donor organs with long times of cold ischemia (Patient A and C) had an excellent outcome of 99% or 95% cell viability, respectively.

### Example 2

### Cell Seeding and Culturing

Alginate scaffolds in 24-well plates (AlgiMatrix™ 3 D Culture System) were purchased from Invitrogen (Carlsbad, Calif., USA, Cat. No. 12684-023). According to the manufacturer, the scaffolds are free of animal-derived compounds and have a pore size of 50-200 μm. Directly before being used, the scaffolds were transferred to a 24-well culture plate with a special ultra-low attachment surface (Corning, Lowell, USA) to minimize cell attachment on the culture plate.

Hepatocytes were resuspended in culture medium to obtain a single cell suspension. The alginate scaffolds were homogenously seeded with a defined volume of 200 μl cell suspension per scaffold containing 1×10⁶ hepatocytes. After seeding, 400 μl culture medium was added per well. Hepatocytes on alginate scaffolds were cultured in supplemented Williams' Medium E without L-Glutamine (Invitrogen, Carlsbad, Calif., USA) as described previously (Bierwolf et al. (2011) Biotechnol Bioeng 108, 141). Cytochrome P450 (CYP) isoenzymes were induced in 2 of the 3 experiments. Accordingly, the culture medium was supplemented from day 3 of cell culture with 2% DMSO, 2 mM 3-methylcholanthrene and 10 mM dexamethasone (Sigma Aldrich, St. Louis, Mo., USA). The scaffolds were incubated under static conditions in a humidified atmosphere of 5% CO₂ and 95% air at 37° C. during a culture period of 14 days. Culture medium was changed every 24 hours. The supernatant was collected every other day and stored at 4° C. or −20° C. for further analyzes.

The DNA content per scaffold was measured for estimation of hepatocyte leakage from the scaffolds. At day 1, 7 and 14 of cell culture, the scaffolds were dissolved according to the manufacturer's guidelines. 1 ml of warm (37° C.) AlgiMatrix™ Dissolving Buffer (Invitrogen) was added per tube and the tubes were incubated at 37° C. for 10 min. After incubation, the tubes were centrifuged at 200×g for 4 minutes. The supernatant was removed and the procedure was repeated. The released cells/spheroids were washed with Dulbecco's Phosphate Buffered Saline (Invitrogen, Carlsbad, USA) and stored at −80° C. DNA was purified using the QIAamp DNA Mini Kit (Qiagen, Germantown, Md., USA) according to the manufacturer's guideline. For DNA measurements the absorbance at 260 nm was monitored.

Results:

Numerous hepatocytes were found to be immobilized in the scaffold pores immediately after cell seeding. After 24 hours in 3D culture hepatocytes revealed aggregation within the pores of the scaffold. From day 3 onwards formation of spheroids was observed approaching their maximum diameter of nearly 100 μm at day 7.

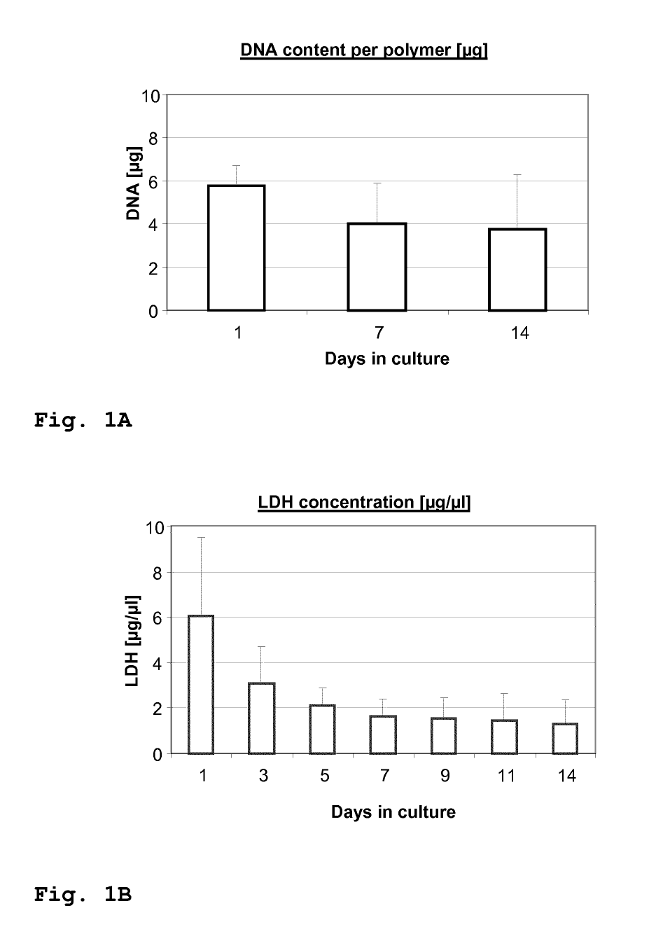

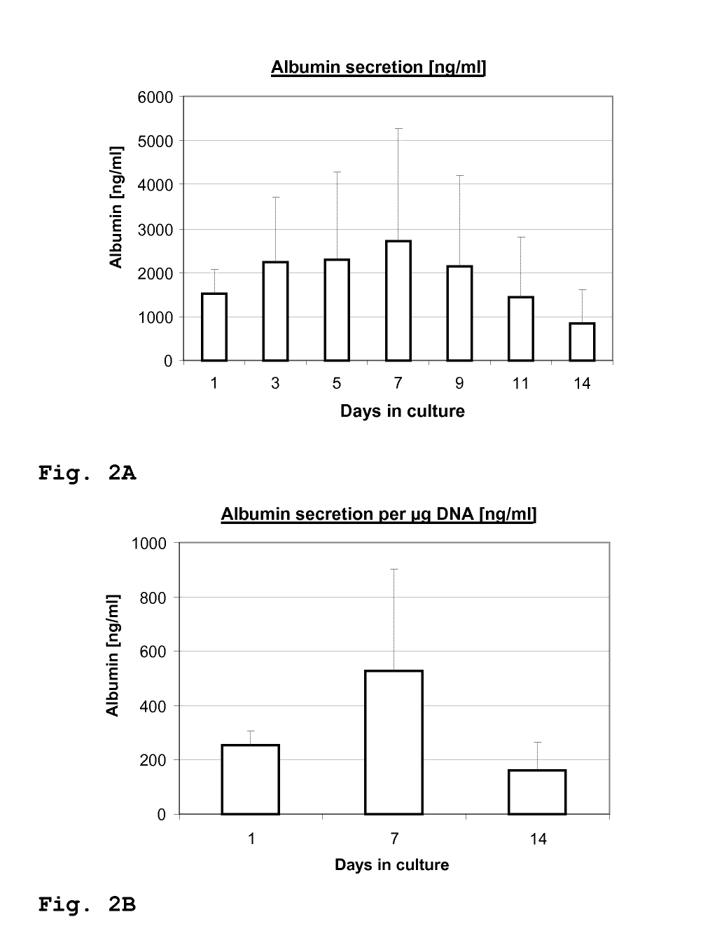

The determination of the DNA content of the scaffolds revealed only a marginal loss of hepatocytes during the 14 day culture period (FIG. 1A). An average amount of 5.79±0.92 μg DNA was determined 1 day after cell seeding. DNA concentration per polymer at day 7 was 4.02±1.85 μg implicating a loss of 30.57% in relation to day 1. At day 14 of 3D cell culture 3.75±2.53 μg DNA was detected per scaffold in 24-well format implicating a loss of 35.23% of cells relative to day 1.

### Example 3

### Lactate Dehydrogenase Assay

Lactate dehydrogenase (LDH) release from damaged cells was monitored in order to determine cell viability and to screen for toxicological effects caused by the scaffolds and their degradation products. Like the other biochemical assays explained below in Examples 4-6, LDH release measuring was performed for every other day during cell culturing using cell-free culture supernatants, i.e. culture medium, that was in contact to the cultured spheroids for the last 24 hours. LDH activity was determined using a Cytotoxicity Detection KitPlus based on colorimetric measurement (Roche, Basel, Switzerland). Incubation time in dark environment was precisely complied with. A LDH standard curve was created via LDH solution from hog muscle (Roche). Colour reactions at 490 nm and 690 nm were monitored, and LDH quantity was calculated in relation to standard curve and in consideration of dilution and background.

Results:

LDH release from damaged cells decreased from 6.05±3.49 μg/μl at day 1 to 2.10±0.79 μg/μl at day 5 and remained almost constant thereafter on very low levels until the end of cell culture (FIG. 1B). This indicates that the cultured cells suffered only from minor cellular membrane damage.

### Example 4

### Human Albumin Assay

Human albumin concentration was measured by enzyme-linked immunosorbent assay (ELISA) using a human albumin quantification kit (ICL, Newberg, Oreg., USA). The absorbance at 450 nm was monitored for each sample in relation to a standard curve. Albumin quantity was interpolated from standards corrected for sample dilution and background.

Results:

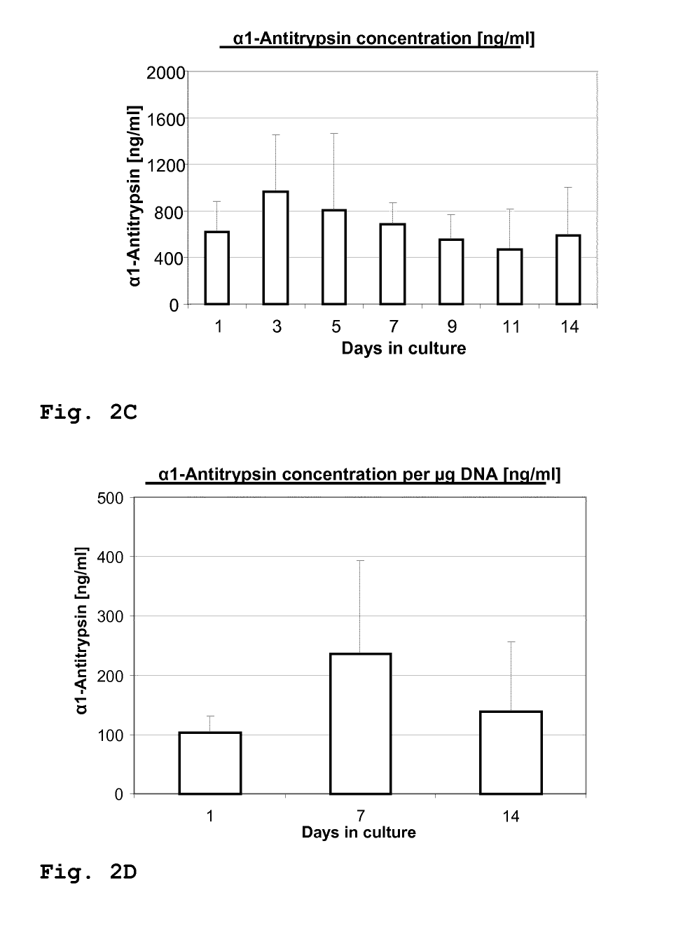

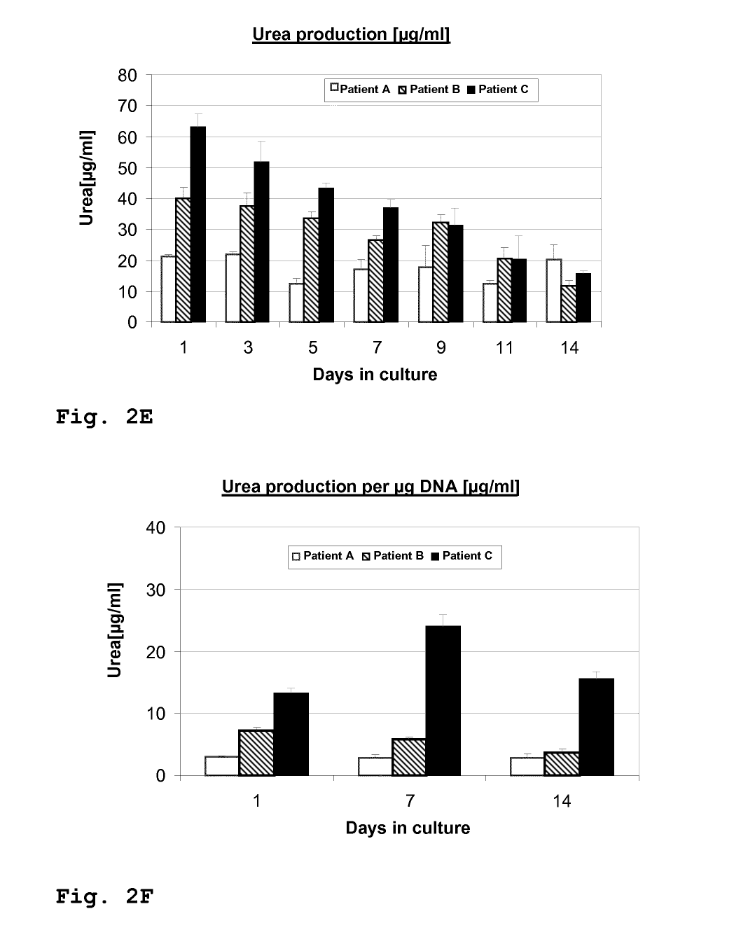

Human albumin concentration in 24 hour supernatant increased from 1513±561 ng/ml at day 1 to a maximum of 2714±2571 ng/ml at day 7 and decreased thereafter to 841±776 ng/ml after 14 days of 3D culture using alginate scaffolds (FIG. 2A). These results were put into relation with the DNA determined in Example 2. Calculating the results from albumin assay to DNA content per scaffold, 24 hour albumin secretion per μg DNA increased from 253±53 ng/ml at day 1 to 528±375 ng/ml at day 7 and declined to a level of 161±104 ng/ml at the end of 3D cell culture (FIG. 2B).

### Example 5

### α1-Antitrypsin Assay

For quantitative determination of α1-antitrypsin in cell culture ELISA-system from Immundiagnostik AG (Bensheim, Germany) was used detecting the hepatic form of α1-antitrypsin. The absorbance was determined at 450 nm and 620 nm. The values of α1-antitrypsin in the samples were calculated from kit containing standards with known concentrations in consideration of dilution and background. A positive and a high positive control were moreover analyzed as quality control.

Results:

Active production of α1-antitrypsin was observed during the entire culture period of 14 days, in which the maximum production rate was measured at day 3, where 965±493 ng protein per ml was detected in 24 hour cell culture supernatant (FIG. 2C). Taking into consideration the DNA content per scaffold, the levels increased from 103±27 ng/ml α1-antitrypsin per μg DNA at day 1 to 236±157 ng/ml per μg DNA at day 7 of cell culture. The values at day 14 declined to 139±118 ng/ml per μg DNA, but they were still higher than the levels measured at day 1 (FIG. 2D).

### Example 6

### Urea Assay

Urea production was determined by a quantitative colorimetric urea assay kit from Biochain Institute (Hayward, Calif., USA). For estimation of urea values in the samples optical density at 430 nm was measured and calculated from urea standard (included in the kit) regarding sample dilution and background.

Results:

Results of urea production were evaluated for every single experiment, due to the different basic disorders. Patients A and B suffered from urea cycle disorders, expecting different values of urea production in comparison to Patient C who suffered from oxalosis. The results from urea assay are demonstrated in FIG. 2E or FIG. 2F, respectively. Hepatocytes from patients A and B exhibited almost constant urea production per μg DNA, but the levels were low during the entire culture period as expected (FIG. 2F). Liver cells from patient C produced higher levels of urea. The maximum level of urea production in relationship to DNA content per scaffold was detected at day 7, where 24±2 μg urea per ml and μg DNA was measured. The values at day 14 (15±1 μg/ml/μg DNA) in this experiment were higher compared to the baseline values at day 1 (13±1 μg/ml/μg DNA).

### Example 7

### Histological Studies

At day 1, 7 and 14 of cell culture scaffolds were embedded in Tissue-Tek (Sakura, Staufen, Germany) and cut into 16 μm thick sections. After fixation in acetone the sections were stained with hematoxylin and eosin (HE) for evaluation of cell viability and with periodic acid Schiff (PAS) for estimation of glycogen storage capacity and analyzed by transmission light microscopy.

Further, the activity of hepatocyte-specific factors was demonstrated by immunofluorescence staining. Hoechst 33258 (Invitrogen) was applied as counterstaining for viable cell nuclei (dilution 1:20000, incubation 1 minute) in all stainings. The sections were visualized by fluorescence microscopy. Heptocyte nuclear factor 4 (HNF-4)/Cytokeratin 18 (CK18) immunofluorescent doublestaining was established to observe the state of cell differentiation. HNF-4 is one of the major liver enriched nuclear hepatocyte transcription factors in normal liver tissue. Sections were incubated for 1 hour with 1:200 diluted HNF-4 goat polyclonal antibody (Santa Cruz Biotechnology, Santa Cruz, Calif.) together with 1:100 diluted CK18 mouse anti human monoclonal antibody (Antibodies-online, Aachen, Germany). As secondary antibodies Alexa Fluor 555-conjugated donkey anti goat (red) and Alexa Fluor 488-conjugated donkey anti mouse (green) antibodies (Invitrogen) were used. Zonula occludens protein 1 (Zo-1) is a marker for tight junctions indicating bile canaliculi formation between flanking hepatocytes in the liver and bipolar configuration. Rabbit polyclonal antibody against Zo-1 was purchased from Invitrogen (1:20, 1 hour). The goat anti rabbit secondary antibody was Alexa Fluor 555-conjugated (red) and manufactured by Invitrogen. For the detection of bile canaliculi the sections were incubated for 1 hour in Alexa 488-labeled Phalloidin (Invitrogen; 1:50, 1 hour), to stain actin filaments (green).

Cytochrome P450 in primary human hepatocytes was stained with rabbit polyclonal primary antibody from MBL International (Woburn, Mass., USA; 1:100, 1 hour) as previously described (Laszlo et al. (2008) Histochem Cell Biol 130, 1005). An Alexa 555-conjugated goat anti rabbit secondary antibody (red) from Invitrogen was used.

Results:

HE staining showed that primary human liver cells cultured on alginate scaffolds maintained high cell viability until day 7 of culture. Furthermore, a well organized cytoskeletal network within the spheroids was demonstrated by immunofluorescent staining of cytokeratin 18. At day 14, a loss of intact cytoskeleton and a decreased viability with central spheroid necrosis were observed by HE and immunofluorescent staining. The PAS reaction demonstrated well preserved glycogen storage in all areas of the hepatocyte spheroids up to 7 culture days. Glycogen storage capacity indicates that the cultured cells are functional hepatocytes. At day 14, single cells within the spheroids remained negative for PAS reaction indicating a loss of function or dedifferentiation, correspondingly.

On day 7, HNF-4 positive hepatocyte nuclei were detected in combination with an in vivo like cytoskeleton of 3D cell culture which indicated a highly preserved cell differentiation. Also, immunofluorescent staining revealed ZO-1 positive hepatocytes within the spheroids at day 7 of culture. ZO-1 is visible between adjacent cells as two parallel stripes defining bile canaliculi. For the detection of actin filaments in bile canaliculi Alexa 488-labeled Phalloidin was used. Reformation of bile canaliculi between the adjacent hepatocytes was observed at culture day 7, displaying liver like fine-structure within the spheroids. With respect to cytochrome P450, immunofluorescent staining revealed positive cells after 7 culture days, indicating the capability to metabolize toxic substances.

It can be concluded from the histological studies that hepatocytes were most suitable for transplantation after 7 days of culturing on the alginate scaffolds.

### Example 8

### Apoptose Assay

At day 7 of cell culture, one scaffold was dissolved according to the manufacturer's guidelines and as described above. The released cells/spheroids were embedded in Tissue-Tek and cut in 8 μm thick sections. Terminal deoxynucleotidyl transferase-mediated dUTP-biotin nick end labeling (TUNEL) reaction was performed using the In Situ Cell Death Detection Kit (Roche) for identification of apoptotic cells (green). The kit was applied according to the manufacturer's guidelines. In addition to TUNEL, immunofluorescent staining of CK18 for cytoskeleton (1:100, 60 min incubation) was performed using an Alexa Fluor 555-labeled goat anti mouse secondary antibody (red) manufactured by Invitrogen (1:800, 45 min incubation).

Results:

The results from the TUNEL reaction with CK18 immunofluorescent staining as background indicated that at day 7 most cells were viable with intact cytoskeleton and nuclei and only some single cells were positive for TUNEL reaction. From this, it can be concluded that the scaffold dissolving procedure is safe and does not induce apoptosis in the hepatocytes.

### Example 9

### RT-PCR and Real-Time-PCR

After harvesting at day 1, 7 and 14 of cell culture the scaffolds were dissolved. The released cells/spheroids were incorporated into 350 μl RLT-buffer (Qiagen) and 3.5 μl β-mercaptoethanol (Sigma-Aldrich). The same process was performed with the native tissue or the cells frozen directly after isolation, respectively. The total RNA was extracted from the lysate using RNeasy Mini Kit (Qiagen). RNA content and purity were determined by absorbance measurement at 260 and 260/280 nm, respectively. For cDNA synthesis the First Strand cDNA Synthesis Kit for RT-PCR (AMV) from Roche was used according to the manufacturer's guidelines. The first strand cDNA synthesis reaction was performed under the following conditions: 25° C. for 10 min, 42° C. for 60 min, 99° C. for 5 min and cooling to 4° C.

Real-time-PCR amplification was deployed to quantify the gene expression of liver cell specific factors by using the QuantiTect SYBR Green PCR Kit (Qiagen, Hamburg, Germany) in combination with gene-specific QuantiTect Primer Assays (Qiagen). The details for the target genes are listed in Table 2. The expression of liver cell specific factors was quantified using the comparative CT method, which calculates the gene expression to an internal housekeeping gene. Human Beta-Actin (ACTB) was used for internal control due to their stable expression. All reactions were performed in duplicates and consisted of 12.5 μl 2× QuantiTect SYBR Green PCR Master Mix, 2.5 μl 10× QuantiTect Primer Assay, and 1 μl cDNA as PCR template. The reactions were performed using the StepOnePlus Real-time-PCR-System (Applied Biosystems, Foster City, Calif., USA). The cycling conditions were as followed: 95° C. for 10 min followed by 45 cycles with 94° C. for 15 sec, 60° C. for 30 sec and 72° C. for 30 sec. Melting curve analysis were performed routinely.

Results:

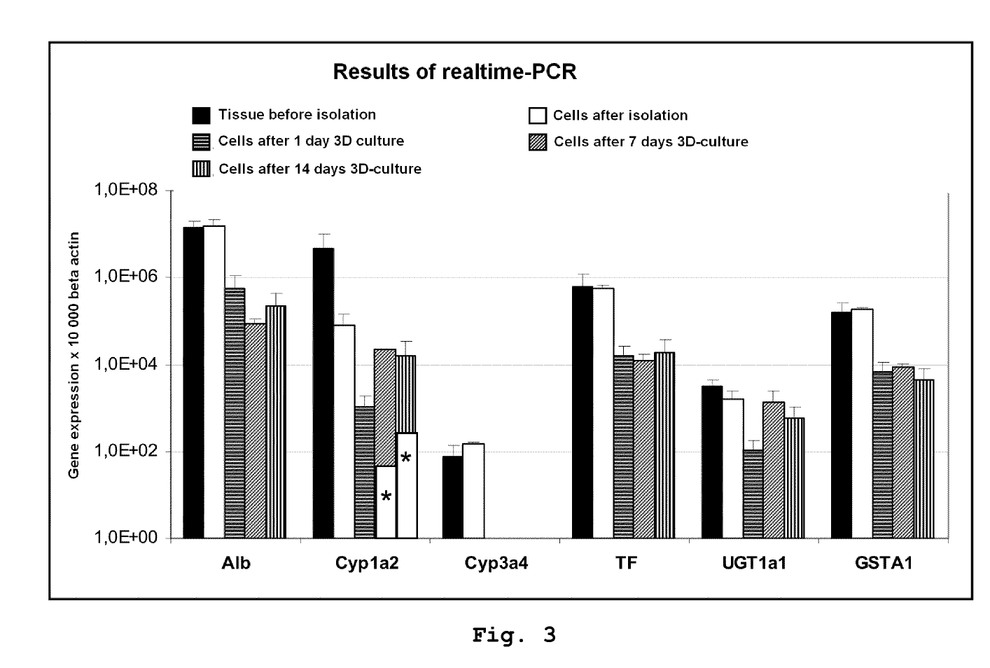

Gene expression levels of liver derived factors have been evaluated by quantitative real-time-PCR. FIG. 3 summarizes the gene expression results of 3D cultured hepatocytes compared to the native tissue as well as the freshly isolated cells. Constant albumin mRNA expression was measured in native tissue and freshly isolated cells, respectively, whereas albumin PCR signal in cultured cells was nearly 10% of the baseline tissue values. The expression of CYP1A2 was strongly influenced by the isolation procedure. The levels in the freshly isolated cells decreased drastically in comparison of the native tissue. CYP P450 enzyme expression was not induced in the experiment using the cells from patient A. CYP1A2 expression level in this experiment was for that reason lower than in the later trials as expected (marked in the figure by an asterisk). CYP3A4 gene expression was already very low in the basic tissue and not detectable in the cultured cells. Copy number of transferrin remained constant during the entire culture period. PCR signal of phase II enzyme UDP glucuronosyltransferase (UGT1A1) decreased at day 1, but recuperated at a later stage of culture. Furthermore, an unaffected expression of phase II enzyme Glutathione S-transferase (GSTA1) was detected.

### Example 10

### In Vivo Transplantation

For the in vivo experiments, primary human hepatocytes were isolated from the livers of 3 patients. Two patients suffered from maple syrup urine disease, and one patient from primary oxalosis. Table 2 provides more details regarding the donors. Spheroids were prepared as described in the above Examples 1 and 2 and transplanted in to uPA/scid mice via the spleen as described in more detail in Dandri et al. (2000), Hepatology 32 (1), 139-146; Dandri et al. (2001), Hepatology 33 (4), 981-988; Dandri et al. (2005), J Hepatology 42 (1), 54-60; Dandri et al. (2008), Hepatology 48 (4), 1079-1086; Petersen et al. (2008), Nature Biotechnology 26 (3), 335-341.

TABLE 2
Details on patients from which primary human hepatocytes
were derived for in vivo testing

| | | | Time of | Cell |
|---|---|---|---|---|
| | Donor | | cold ischemia | Viability |
| Patient | age/sex | Donor diagnosis | (hours) | (%) |
| A | 18 years/m | Primary oxalosis | 48 | 95 |
| B | 4 months/m | Maple syrup urine | 16 | 75 |
| | | disease | | |
| C | 2 months/m | Maple syrup urine | 7 | 99 |
| | | disease | | |

| | |
|---|---|
| Average viability ± Standard deviation (%) | 89.5 ± 10.5 |

Eight weeks after transplantation, the mice were sacrificed, and the liver organs were examined. Immediately prior to sacrificing, serum was taken and tested for human albumin in the ELISA assay described in Example 4. Human albumin can only be produced in cases were the transplanted spheroids have successfully integrated into the murine liver. The organs obtained from the mice were also examined by different histological staining procedures, including human cytokeratin 18, α1-antitrypsin, Zo-1, cytochrome P450 and HNF-4.

Results:

As can be shown in the below tables, seven animals were positive for human hepatocytes eight weeks after transplantation. In three animals tested from this group, human albumin was detected in the serum of the recipient mice which shows that the transplanted spheroids have successfully integrated into the murine liver tissue.

TABLE 3
Results from transplantation experiments

| | | Numbers of | Mice positive | Human |
|---|---|---|---|---|
| | 3D pre-culture | transplanted | for human | albumin in |
| Patient | (days) | mice | hepatocytes | serum (mg/ml) |
| A | 7 | 5 | 1 | 0.497 |
| B | 7 | 5 | 2 | 1.312 |
| | | | | 1.396 |
| C | 6 | 5 | 4 | Not analyzed |

| | | |
|---|---|---|
| Total animals | 15 | 7 |

These results were further supported by the results of the histological experiments. Here, it could be shown that the human hepatocytes started to proliferate after integration into the mouse liver and formed clusters that could be visualized by staining for human cytokeratin 18. The formation of tight junctions and gap junctions was detected by staining for human Zo-1 and human connexion 32, respectively.

Additional histological staining procedures revealed that the hepatocytes that have been integrated into murine liver tissue produced human α1-antitrypsin and the liver-specific factor HNF-4. The presence of new bile canaliculi within the transplanted tissue was visualized by staining with phalloidin. The functional capability of the human hepatocytes in the mouse liver to degrade toxic compounds was demonstrated by staining for cytochrome P450.

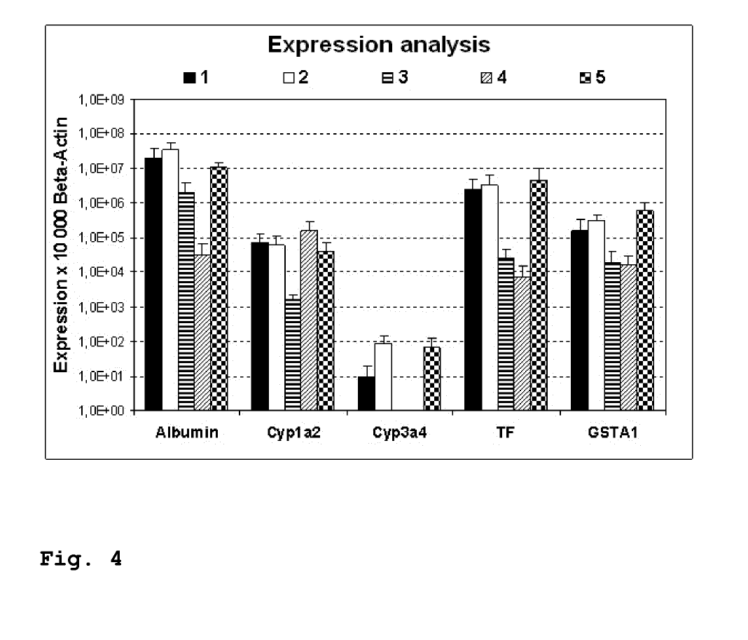

The expression of liver-specific genes in the murine liver was detected by real-time-PCR. The results are shown in FIG. 4. The primers used in the PCR analysis were designed to detect exclusively human transcripts. The results of the PCR demonstrate that the expression of liver-specific genes is down-regulated compared to human native liver tissue when cells are cultures to spheroids in vitro; however, when transplanted into the murine liver, the initial expression levels are reached again. In some cases, the expression levels detected after transplantation into mice were higher than those in the native human liver tissue.

1. Isolated hepatocyte spheroid of cultured human primary hepatocytes, wherein said spheroid is free of any artificial scaffold material.

2. Isolated spheroid of claim 1, wherein the production of human α1-antitrypsin in the hepatocytes of said spheroid is at least 50% of the production determined in freshly isolated human primary hepatocytes.

3. Isolated spheroid of claim 1, wherein the production of human albumin in the hepatocytes in said spheroid is at least 50% of the production determined in freshly isolated human primary hepatocytes.

4. Isolated spheroid of claim 1, wherein bile canaliculi between adjacent hepatocytes are present in at least 50% of the hepatocytes in said spheroid.

5. Isolated spheroid of claim 1, wherein said spheroid has a diameter of at least 50 μm.

6. A liver cell transplantation medicine comprising an isolated spheroid of claim 1.

7. A method of treating liver disease in a patient having diseased liver tissue, said method comprising transplanting the isolated spheroid of claim 1 into the diseased liver tissue of said patient.

8. The method of claim 7, wherein said liver disease is selected from the group of hepatitis A, B and C, liver cirrhosis, α1-antitrypsin deficiency, Wilson Disease, hemochromatosis, bile duct obstruction, glycogen storage disease, Reye's syndrome in young children, hereditary tyrosinemia type I, parasitic infections, primary sclerosing cholangitis, secondary sclerosing cholangitis, chronic Budd Chiari syndrome, polycystic liver disease, oxalosis, urea cycle defects, mitochondrial depletion syndrome, Alagille syndrome, Crigler-Najjar syndrome, primary familiar intra-hepatic cholestasis, neonatal hepatitis, biliary atresia, fulminate hepatic failure, alcoholic cirrhosis, autoimmune hepatitis, overlap syndrome, liver intoxication due to paracetamol, phalloidin or other agents.

9. The method of claim 8, wherein said spheroid is derived from primary hepatocytes which are autologous to the patient.

10. Method for preparing a spheroid of human primary hepatocytes, comprising the steps of:
(a) culturing isolated human primary hepatocytes on a polysaccharide scaffold under conditions that allow the formation of a hepatocyte spheroid;
(b) dissolving the polysaccharide scaffold to release the hepatocyte spheroid; and
(c) separating the hepatocyte spheroid from the culture medium.

11. Method of claim 10, wherein said polysaccharide scaffold is an alginate scaffold.

12. Method of claim 11, wherein said alginate scaffold is dissolved in step (b) by the addition of citric acid or EDTA.

13. Method of claim 10, wherein separating the hepatocyte spheroid in step (d) comprises filtration and/or centrifugation.

14. Method of claim 10, wherein said culturing in step (a) is performed for 7-14 days.

15. Isolated hepatocyte spheroid of human primary hepatocytes obtainable by a method of claim 10.

16. (canceled)
